(19) United States
(12) Patent Application Publication
(10) Pub. No.: US 2026/0256794 A1
(43) Pub. Date: Sep. 3, 2026

(54) Oral Dynamine Formulations and Methods Therefor

(71) Applicants: Matthew Titlow, Carlsbad, CA (US); Tim N. Ziegenfuss, Chardon, OH (US); Ortho-Nutra, LLC, Cream Ridge, NJ (US)

(72) Inventors: Matthew Titlow, Carlsbad, CA (US); Tim N. Ziegenfuss, Chardon, OH (US); Hector L. Lopez, Cream Ridge, NJ (US)

(73) Assignee: Ortho-Nutra, LLC, Cream Ridge, NJ (US)

(21) Appl. No.: 19/491,202

(22) PCT Filed: Jun. 7, 2024
(86) PCT No.: PCT/US2024/033015

§ 371 Date: Dec. 8, 2025

Related U.S. Application Data
(60) Provisional application No. 63/507,288, filed on Jun. 9, 2023.

(51) Int. Cl. A61K 31/522; A61K 9/00; A61K 45/06; A61P 25/28

(52) U.S. Cl. CPC A61K 31/522; A61K 9/0053; A61K 45/06; A61P 25/28

(57) ABSTRACT

Orally administered compositions comprising methylliberine as sole active ingredient are presented that desirably modulate one or more of energy, sustained energy, mental stamina, ability to focus, ability to concentrate, motivation to accomplish difficult tasks, drive, vigor, level of positive outlook, mood, feelings of well-being, and ability to tolerate stress.

This application claims priority to our copending US provisional patent application with the Ser. No. 63/507,288, which was filed Jun. 9, 2023, and which is incorporated by reference herein.

### FIELD OF THE INVENTION

The field of the invention is dietary supplements, especially as it relates to compositions comprising DYNAMINE™ (methylliberine, 2-methoxy-1,7,9-trimethylpurine-6,8-dione) as a single active ingredient for oral administration.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

It is known in the art that methylliberine in combination with caffeine or theacrine or a mixture of both may have certain beneficial neurological effects. For example, Yates et al. explore the pharmacokinetic interaction potential of methylliberine (2-methoxy-1,7,9-trimethylpurine-6,8-dione, DYNAMINE™), caffeine, and theacrine in healthy subjects. The article found that methylliberine, when administered in combination with caffeine, will alter pharmacokinetic effects of caffeine. This finding was obtained based on an experiment where subjects were randomized in double blind manner to receive a single oral dose of either methylliberine 25 mg (Cohort I), methylliberine 100 mg (Cohort II), caffeine 150 mg, methylliberine 100 mg and caffeine 150 mg (Cohort III) or methylliberine 100 mg and theacrine 50 mg (Cohort IV). Notably, the results revealed that among all tested compounds methylliberine was rapidly absorbed from the oral administration, with Cₘₐₓ reaching on average at 0.6 and 0.9 hours after following lower (25 mg) and higher (100 mg) doses of methylliberine, respectively, and methylliberine was eliminated with a half-life averaging 1 to 1.4 hours.

Similarly, further reference is made to 2021 Tartar et al. Cureus 13 (12): e20534. DOI 10.7759/cureus.20534. Here, the reference studies the effects of caffeine (125 mg), caffeine (125 mg)+methylliberine (75 mg)+theacrine (50 mg) (CDT), and matched placebos, particularly when administered to male “e-gamers.” The experiment concluded that CDT—the combination of caffeine, methylliberine, and theacrine-boosted attention to internal processing (i.e., increased cortical delta power) and potentially increased cognitive control (i.e., increased cortical theta frequency), and cortisol.

Finally, reference is made to U.S. Ser. No. 11/583,534 that teaches an enhanced caffeinated beverage composition that includes a caffeinated drink combined with an effective blend of methylliberine and theacrine providing increased mood, energy, alertness, focus, motivation, and/or decreased fatigue without adversely affecting heart rate or blood pressure.

As exemplified by the references cited above, even though the prior art references contain various compositions and methods of administering methylliberine in combination with the methylxanthine caffeine or the methylurate theacrine to produce neurological benefits, they fail to explore the neurological benefits conferred by methylliberine alone. Therefore, there remains a need for improved methylliberine-containing compositions and methods using same, particularly where methylliberine is the sole active ingredient with regard to neuroenergetic effects.

### SUMMARY OF THE INVENTION

The inventive subject matter is directed to improved methylliberine-containing compositions and methods that modulate various neuroenergetic metrics in a subject, particularly where methylliberine is given as the sole nootropic ingredient.

In one aspect of the inventive subject matter, the inventor contemplates a method of modulating a neuroenergetic metric in a subject that includes a step of administering to the subject a composition comprising methylliberine in an amount effective to modulate the neuroenergetic metric.

Most preferably, administering is orally administering, for example, in combination with a pharmaceutically or nutritionally acceptable carrier. Therefore, suitable compositions can be formulated as a capsule, a powder, a buccal pouch, a nasal spray, or a tablet, or as a ready-to-drink beverage, a tea, an energy drink, or a juice.

In further contemplated aspects, the methylliberine is the sole active ingredient with neuroenergetic function in the composition, or the composition may further comprise an additional active ingredient with neuroenergetic function other than caffeine and/or theacrine.

As will be appreciated, especially contemplated neuroenergetic metrics include energy, sustained energy, mental stamina, ability to focus, ability to concentrate, motivation to accomplish difficult tasks, drive, vigor, level of positive outlook, mood, feelings of well-being, and/or ability to tolerate stress. Most notably, the peak of modulation of the neuroenergetic metric is at least 120 minutes, or even at least 180 minutes post oral administration.

Viewed from a different perspective, the inventor therefore also contemplates a composition for modulating a neuroenergetic metric in a subject. Most typically, such composition will comprise methylliberine in combination with a pharmaceutically or nutritionally acceptable carrier and formulated for oral administration, wherein the methylliberine is present in a dosage in an amount effective to modulate the neuroenergetic metric. With respect to preferred formulations, neuroenergetic metrics, additional ingredients, and time to peak, the same considerations as noted above apply.

Various objects, features, aspects, and advantages of the inventive subject matter will become more apparent from the following detailed description of preferred embodiments.

### DETAILED DESCRIPTION

The inventor has discovered various compositions and methods of improving selected indices of neurological and neuroenergetic functions by administering daily dosages of methylliberine, preferably as the sole nootropic ingredient. In some aspects of the inventive subject matter, such indices were measured by having subjects complete a daily Stroop and Trail Making Test (TMT), and by having subjects fill out anchored Visual Analog Scales (VAS) that assess perceived changes in energy, sustained energy, mental stamina, focus, concentration, motivation, drive, vigor, positivity, mood, well-being, and ability to tolerate stress at 0, 60, 120, and 180 min post-administration.

For example, the applicant discovered that when a subject's cognitive abilities were assessed over time using a Stroop and Trail Making Test (TMT), a placebo group experienced a non-significant increase (+4.4%, p=0.15) in TMT performance, whereas a group treated for 4-days with daily methylliberine supplementation significantly improved in TMT performance (+11.1%, p<0.001). In other examples, the applicant discovered that that, compared to placebo, methylliberine significantly increased energy (En) and sustained energy (SusEn) at 60 min (En: d=0.81, p=0.01, SusEn: d=0.86, p=0.03) and 120 min (En: d=0.69, p=0.04, SusEn: d=0.62, p=0.04) in females. In further examples, the applicant discovered that, compared to placebo, methylliberine ingestion improved a subject's concentration at 180 min (d=0.68, p=0.006), mood at 60 min (d=0.43, p=0.05) and 180 min (d=0.65, p=0.03), well-being at 180 min (d=0.52, p=0.05), and the ability to tolerate stress at 180 min (d=0.54, p=0.04) in both sexes.

These and selected other results (shown in more detail below) were particularly unexpected in view of the known pharmacokinetic data for methylliberine that have shown a very short time to Cₘₐₓ and a short half-life time for orally administered methylliberine. Indeed, it is rather surprising that certain neuroenergetic effects increased or peaked at or after 180 minutes while the Cₘₐₓ of methylliberine is 0.6-0.9 hours, and the half-life is 1-1.4 hours. Viewed from a different perspective, the inventor observed that numerous neuroenergetic benefits increased or peaked at a time (e.g., at or after 180 minutes) at which the skilled artisan would have expected the plasma concentration of methylliberine (and with that the neuroenergetic effects) to have already significantly dropped. While not wishing to be bound by any theory or hypothesis, The applicant contemplates that this unexpected discovery could be the result of a metabolite of methylliberine, or either a synergistic or antagonist interaction between methylliberine (or its metabolite) and a further metabolite, enzyme, or signaling element in a signal transduction pathway. The applicant also contemplates that this unexpected discovery could be the result of indirect effects on the human endocrine system.

In particular, the applicant found that, despite methylliberine absorbing and reaching Cₘₐₓ on average at 0.6 and 0.9 hours after following low (25 mg) and high (100 mg) doses of methylliberine, respectively, and despite methylliberine being eliminated with a half-life averaging 1 to 1.4 hours, test subjects reported significant improvements in select neurological or neuroenergetic improvements through VAS, particularly at 180 min post-ingestion and beyond.

As shown in more detail below, Applicant contemplates that, in certain embodiments of the inventive subject matter, administration of a single dosage of about 100 mg (e.g., between 1.0 and 1.4 mg/kg, or between 1.1 and 1.3 mg/kg) methylliberine over one or more days as single ingredient improves TMT performance as well as various indices of affect (e.g., energy, mood, well-being, ability to tolerate stress). In other embodiments, Applicant contemplates that similar improvements in TMT performance as well as various other ndices of affect can be achieved using dosages of at least about 25 mg, about 50 mg, about 75 mg, about 125 mg, about 150 mg, about 200 mg, about 250 mg, about 300 mg, about 350 mg, about 400 mg, about 450 mg, about 500 mg, or in some cases even larger amounts of methylliberine. As such, suitable quantities therefore include ranges between 10-50 mg, or 50-250 mg, or 250-500 mg, or 500-750 mg, or 750-1,000 mg, and even higher. However, in further embodiments, typical dosages will be less than 10 g, or less than 5 g, or less than 2.5 g, or less than 1 g, or less than 500 mg, or less than 300 mg, and even less.

In addition, it was observed that selected neuroenergetic responses appeared to be dose-effective. Based on the data below (and other considerations not shown here), methylliberine appeared to be most effectively dosed at on a mg per kg of body weight basis, and particularly between 1.0 and 1.4 mg/kg, or between 1.1 and 1.3 mg/kg. Moreover, and with regard to the timing of the observed effects it should be appreciated that positive responses to caffeine (chemically closely related to methylliberine) decline after two hours, with a typical a decrease in mood, concentration, energy, motivation and cognitive performance metrics, despite caffeine's half-life being significantly longer than that of methylliberine. Such findings were entirely unexpected.

After discovering that subjects unexpectedly experience improvements in TMT performance as well as various other select indices of affect at 180 min post-ingestion, Applicant further contemplates that improvements in TMT performance as well as various other select indices of affect in subjects treated with methylliberine would be observed at 200 min, 220 min, 240 min, 260 min, 280 min, 300 min, 320 min, 330 min, 360 min, or in some cases even larger periods of time post-ingestion. As such, suitable periods of time before observing such improvements post-ingestion include 180-200 min, 200-220 min, 220-240 min, 240-260 min, 260-280 min, 280-300 min, 300-320 min, 320-340 min, 340-360 min, 360-380 min, 380-400 min, 400-500 min, 500-600 min, and even longer. However, in further embodiments, typical periods of time before observing aforementioned improvements will be in less than 600 min, or less than 400 min, or less than 300 min, or less than 200 min, or less than 190 min, or less than 185 min post-ingestion.

While subjects in certain embodiments of the inventive subject matters experience improvements in TMT performance as well as certain indices of affect at 180 min post-ingestion, the applicant contemplates that such improvements could be observed in less than 180 min post-ingestion. Applicant further contemplates that such improvements can be observed at 60 min, 70 min, 80 min, 90 min, 100 min, 110 min, 120 min, 130 min, 140 min, 150 min, 160 min, or 170 min post-ingestion. As such, suitable periods of time before observing such improvements post-ingestion include 60-80 min, 80-100 min, 100-120 min, 120-140 min, 140-160 min, 160-180 min, and even longer. However, in further embodiments, typical periods of time before observing aforementioned improvements will be more than 60 min, or more than 80 min, or more than 100 min, or more than 120 min, or more than 140 min, or more than 160 min, or more than 180 min post-ingestion.

Applicant further contemplates that the possible periods of time it will take after ingestion for methylliberine to cause improvements in neuroenergetic functions may be dependent on a subject's metabolism. This could present further experimentation which compare subjects of different age groups, muscle-to-fat ratios, amount of physical activity, hormone function, or other factors (such as CYP450 variants) that could affect metabolism.

In some embodiments of the invention, Applicant contemplates administering a composition containing methylliberine wherein methylliberine makes up at least 50% of the weight of the composition being administered, and typically (but not necessarily) wherein the remaining weight of the composition does not contain caffeine or theacrine. Applicant additionally contemplates administering a composition containing methylliberine wherein methylliberine makes up at least roughly 55%, 60%, 65%, 70%, 75%, 80%, 85%, 90%, 95%, or 99% of the weight of the composition, and wherein the remaining weight of the composition typically (but not necessarily) does not contain caffeine or theacrine or a combination of caffeine and theacrine. As such, suitable percentages of the total weight of the composition consisting of methylliberine therefore include 50-60%, 60-70%, 70-80%, 80-90%, 90-99%, or even higher. However, in further embodiments of the invention, typical quantities will be above 50% of the total weight of the composition.

In further embodiments of the invention, Applicant contemplates administering a composition containing methylliberine wherein methylliberine makes less than 50% of the weight of the composition being administered, and wherein the remaining weight of the composition preferably does not contain caffeine or theacrine. Applicant additionally contemplates administering a composition containing methylliberine wherein methylliberine makes up at least roughly 40%, 30%, 20%, 10%, or even less of the total weight of the composition. As such, suitable percentages of the total weight of the composition consisting of methylliberine therefore include 0.1-10%, 10-20%, 20-30%, 30-40%, and 40-50%. However, in still further embodiments, typical quantities will be above 50% of

In further embodiments of the invention, Applicant contemplates administering methylliberine in combination with a pharmaceutically or nutraceutically acceptable carrier excluding caffeine or theacrine or a combination of caffeine and theacrine. However, metabolites or modified forms of caffeine and/or theacrine are particularly contemplated. Moreover, other ingredients, and especially nootropic ingredients or ingredients affecting neuroenergetic parameters (e.g., sleep, mood, anxiety, focus, etc.) are also contemplated to be included in the formulations presented herein, and particularly preferred ingredients include minerals, vitamins, herbs, herbal extracts, and isolated chemical compounds. For example, contemplated nootropic ingredients include theobromine, resveratrol, phosphatidyl serine, Ginseng extracts or plant parts, theanine, Gingko extracts or plant parts, Rhodiola extracts or plant parts, acetyl-L-carnitine, Bacopa monnieri extracts or plant parts, etc.

Applicant contemplates that in some embodiments of the invention, methylliberine can be administered orally, intravenously, buccally, sublingually, by topical application to the skin, by inhalation or intranasal application, or transdermally via a patch.

### Experiments

The inventors have discovered various compositions and methods of improving various indices of neurological and neuroenergetic functions by administering 100 mg (once daily) dosages of DYNAMINE™ (dosage between 1.0 and 1.4 mg/kg) as described in the following:

Description of Dataset: A blinded dataset of 25 cases (Female: n=13; Male: n=12) was received. All variables listed below were assessed as part of screening: Body Mass, Height, Body Mass Index, Systolic Blood Pressure, Diastolic Blood Pressure, Resting Heart Rate, White Blood Cell Count, Red Blood Cell Count, Hemoglobin, Hematocrit, Glucose, Blood Urea Nitrogen (BUN), Creatinine, BUN: Creatinine, Sodium, Potassium, Chloride, Carbon Dioxide, Calcium, Total Protein, Albumin, Globulin, Albumin: Globulin, Bilirubin, Alkaline Phosphatase, AST, ALT, Total Cholesterol, Triglycerides, HDL Cholesterol, VLDL Cholesterol, LDL Cholesterol, LDL: HDL, Energy Intake, Carbohydrate Intake, Fat Intake, Protein Intake, and body mass. Table 1 is a summary of exemplary data of the subjects enrolled in the studies.

TABLE 1

| | | Mean | (SD) |
|---|---|---|---|
| Age | Female | 33.5 | (11.1) |
| (years) | Male | 33.5 | (10.7) |
| | Total | 33.5 | (10.7) |
| Height | Female | 66.2 | (2.8) |
| (inches) | Male | 70.0 | (3.0) |
| | Total | 68.0 | (3.4) |
| Body Mass | Female | 149.8 | (24.4) |
| (pounds) | Male | 173.7 | (17.7) |
| | Total | 161.3 | (24.3) |
| Body Mass Index | Female | 23.9 | (2.6) |
| (kg/m²) | Male | 24.9 | (1.4) |
| | Total | 24.4 | (2.2) |
| Systolic Blood Pressure | Female | 113.3 | (13.5) |
| (mm Hg) | Male | 122.4 | (8.7) |
| | Total | 117.7 | (12.1) |
| Diastolic Blood Pressure | Female | 76.5 | (7.8) |
| (mm Hg) | Male | 78.6 | (9.8) |
| | Total | 77.5 | (8.7) |
| Resting Heart Rate | Female | 70.0 | (12.6) |
| (beats/min) | Male | 66.2 | (8.7) |
| | Total | 68.2 | (10.8) |

All neuroenergetic tests as listed below were performed and data were collected after 0 and 4 days of assigned supplements (with acute effects also measured at day 4 over 180 minutes):

Stroop Color Matching Test (Total Score, Accuracy %, Average Score): Day 0: Two blocks of three tests were completed. Each block of three tests was separated by 10 minutes. The median of each block was taken and then those two scores were averaged. This value was used in all statistical analysis. Day 4: Three tests were completed at four time points: after 0, 60, 120, and 180 minutes of taking dose #4. The median of the three tests at each time point was used in all statistical analysis.

Trail Making Test: Day 0: Two blocks of three tests were completed. Each block of three tests was separated by 10 minutes. The median of each block was taken and then those two scores were averaged. This value was used in all statistical analysis. Day 4: Three tests were completed at four time points: after 0, 60, 120, and 180 minutes of taking dose #4. The median of the three tests at each time point was used in all statistical analysis.

All VAS Measurement (Energy, Sustained Energy, Mental Stamina, Ability to Focus, Ability to Concentrate, Motivation to Accomplish Difficult Tasks, Drive, Vigor, Level of Positive Outlook, Mood, Feelings of Well-Being, Tolerate Stress); Day 0: Two tests were completed. The average of both scores was computed and this value was used in all statistical analysis. Day 4 (0 minutes after ingesting dose #4): Two tests were completed. The average of both scores was computed and this value was used in all statistical analysis. Day 4 (60, 120, and 180 minutes after ingesting dose #4): One test was completed at each time point.

Hemodynamics Systolic Blood Pressure, Diastolic Blood Pressure, Heart Rate were taken Pre and Post Stroop Test on Day 0; Pre and Post Stroop Test after 4 Days of Supplementation 0, 60, 120, and 180 minutes after ingestion of 4th dose.

Statistical Approach Employed: A significance level of 0.05 was used for all statistical determinations. P-values between 0.051 and 0.10 were deemed a trend/tendency. All variables were tested for normality using results from a Shapiro-Wilk test. When a deviation from normality was identified, traditional transformation approaches were employed. In case of scale (ratio) data, transformations were not possible. Parametric data are presented as means+standard deviation and analyzed using t-tests and ANOVAs. Variables that grossly deviated from normality are presented as mean±standard deviation and mean ranks and analyzed first using the Friedman test (within-group changes), then the Wilcoxon signed rank test (paired differences within group) and then Mann-Whitney U test (between-group differences).

Participants deemed non-compliant were dropped from the dataset prior to the analysis. For all non-baseline missing datapoints, data was replaced by carrying the last observed datapoint forward. All efficacy outcomes were then analyzed using a ‘per protocol’ approach. Factorial ANOVA with repeated measures on time were used to examine changes from baseline within each group. Mixed factorial ANOVA with repeated measures on time were used to assess group, time, and group×time interaction effects. Changes from baseline were calculated and independent t-tests were computed to evaluate between-group changes. Area under the curve (AUC) was computed with Microsoft Excel using the trapezoidal rule and subsequently analyzed using paired samples t-test. All paired compared comparisons between groups were evaluated using mean difference #SE, 95% confidence intervals on the differences between groups and effect sizes. The tables and figures provided herein highlight within-group and between group changes. Area Under the Curve Analysis: No between-group differences were identified for any of the visual analog scale area under the curve calculations.

Summary of outcomes: The following summaries provide the outcomes of the studies performed for the exemplary neuroenergetic metrics where Group B denotes methylliberine and where Group A denotes placebo, unless noted otherwise.

### Visual Analog Scales (VAS)

Neuroenergic parameters are inherently subjective, nevertheless significant in human behavior and well-being. To allow for a standardization among different subjects, the following experiments were performed using a visual analog scale using anchor statements (lowest possible/highest possible) in which subjects were asked to rate their subjective experience with respect to the specific neuroenergic parameters tested.

VAS-Energy: In this subjective test, subjects were asked to rate their personal level of energy on a VAS scale using the prompt of “My level of energy right now is . . . ” (with rating lowest possible to highest possible)”. Results were collected as noted above and Table 2 provides exemplary results for Day 0 versus Day 4 ratings, whereas Table 3 provides exemplary acute results measured at Day 4 over a duration of 180 minutes. Table 4 shows changes from baseline.

TABLE 2
Day 0 vs Day 4

| | | Group A | | Group B | | Between-Group | |
|---|---|---|---|---|---|---|---|
| Variable | | Mean | (SD) | Mean | (SD) | Effects | |
| | Day 0 | 5.6 | (1.9) | 5.5 | (2.0) | Group | 0.84 |
| | Day 4 | 5.6 | (1.9) | 5.8 | (2.1) | Time | 0.26 |

| | | | | |
|---|---|---|---|---|
| Within | 0.95 | 0.16 | Group × Time | 0.27 |
| Group (p) | | | | |

TABLE 3
Day 4: 0, 60, 120, and 180 minutes

| | | A | | B | | Between-Group | |
|---|---|---|---|---|---|---|---|
| Variable | Time | Mean | (SD) | Mean | (SD) | Effects | |
| | 0 | 5.6 | (1.9) | 5.8 | (2.1) | Group | 0.14 |
| | 60 | 5.8 | (2.0) | 6.2 | (1.9) | Time | 0.001 |
| | 120 | 6.1 | (1.8) | 6.2 | (2.2) | Group × | 0.23 |
| | | | | | | Time | |
| | 180 | 6.1 | (1.9)* | 6.5 | (1.9)* | | |

| | | |
|---|---|---|
| Within | 0.02 | 0.01 |
| Group (p) | | |

*= Different from 0-minute time point (p < 0.05)

TABLE 4
Changes from baseline

| | Mean Difference ± SE | p | 95% CI | d |
|---|---|---|---|---|
| 60 min-0 min | 0.24 ± 0.22 | 0.29 | (−0.22, 0.70) | 0.28 |
| 120 min-0 min | −0.14 ± 0.27 | 0.61 | (−0.69, 0.41) | −0.14 |
| 180 min-0 min | 0.28 ± 1.24 | 0.28 | (−0.23, 0.79) | 0.28 |

Notably, the group×sex×time interaction tended to be different (p=0.064) for VAS Energy ratings. This means that the two sexes responded differently between the two supplemental conditions across time. To effectively decompose this interaction, separate 2×4 (group×time) mixed factorial ANOVA with repeated measures on time were completed separately for each gender. For males, no significant group×time interaction was observed for the males (p=0.81), however unexpectedly, for females a significant group×time interaction was observed for the females (p=0.020). Table 5 shows further data depicting mean differences.

TABLE 5
Mean Differences

| | | | | Mean | | | |
|---|---|---|---|---|---|---|---|
| | Group | Mean | (SD) | Difference ± SE | p | 95% CI | d |
| Δ @ 60 min | A | −0.06 | (0.87) | 0.72 ± 0.23 | 0.01 | (0.21, 1.23) | 0.81 |
| | B | 0.66 | (0.90) | | | | |
| Δ @ 120 min | A | 0.45 | (0.63) | −0.03 ± 0.38 | 0.94 | (−0.85, 0.79) | −0.03 |
| | B | 0.42 | (1.45) | | | | |
| Δ @ 180 min | A | 0.35 | (0.74) | 0.73 ± 1.11 | 0.04 | (0.06, 1.40) | 0.69 |
| | B | 1.08 | (1.31) | | | | |

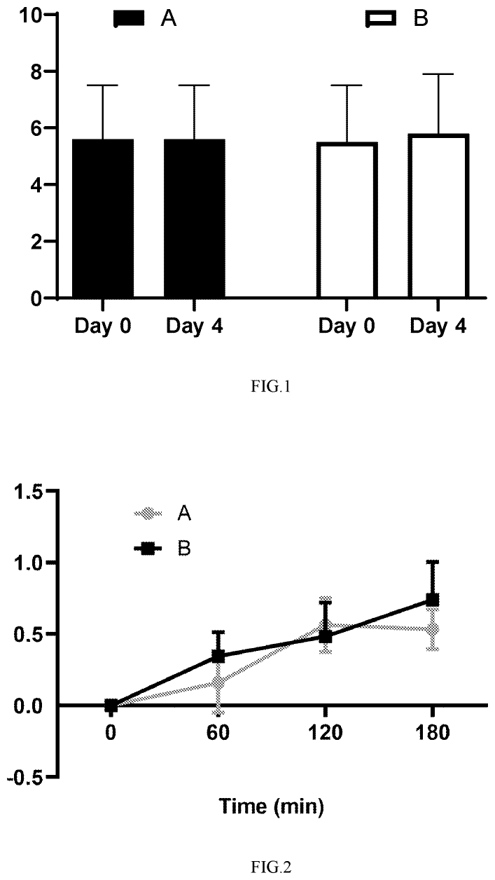

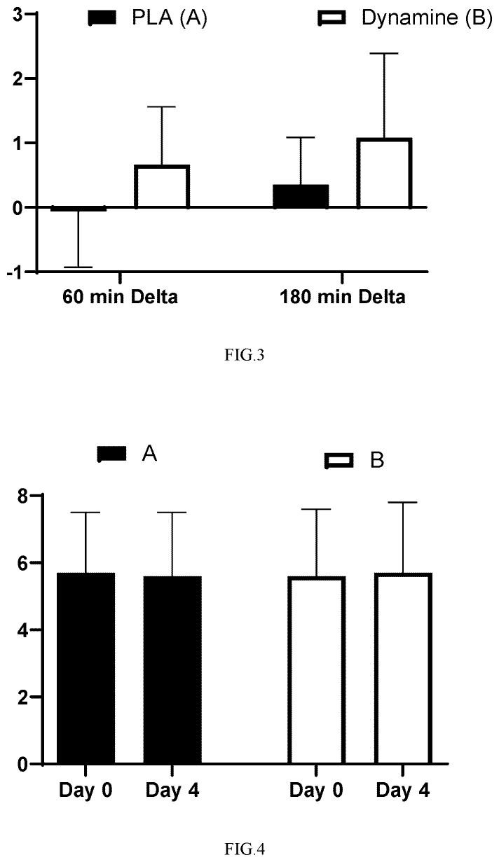

Based on the above, it can therefore clearly be seen that for Day 0 vs. Day 4 no significant group×time interaction (p=0.27) existed as is also graphically shown in FIG. 1. Similarly, at Day 4 (0-minute vs. 60, 120, and 180 minutes) no significant group×time interaction (p=0.23) was observed as is also graphically shown in FIG. 2. Notably, the 3-way gender×condition×time interaction tended to be significant (p=0.064). Here, for males no significant group×time interaction was observed for (p=0.68), whereas for females a significant group×time interaction was observed for female (p=0.022).

VAS-Sustained Energy: In this subjective test, subjects were asked to rate their personal level of energy on a VAS scale using the prompt of “My level of sustained energy right now is . . . ” (with rating lowest possible to highest possible)”. Results were collected as noted above and Table 6 provides exemplary results for Day 0 versus Day 4 ratings, whereas Table 7 provides exemplary acute results measured at Day 4 over a duration of 180 minutes.

TABLE 6
Day 0 vs Day 4

| | | Group A | | Group B | | Between-Group | |
|---|---|---|---|---|---|---|---|
| Variable | | Mean | (SD) | Mean | (SD) | Effects | |
| | Day 0 | 5.7 | (1.8) | 5.6 | (2.0) | Group | 0.75 |
| | Day 4 | 5.6 | (1.9) | 5.7 | (2.1) | Time | 0.97 |

| | | | | |
|---|---|---|---|---|
| Within | 0.48 | 0.50 | Group × Time | 0.38 |
| Group (p) | | | | |

TABLE 7
Day 4: 0, 60, 120, and 180 minutes

| | | A | | B | | Between-Group | |
|---|---|---|---|---|---|---|---|
| Variable | Time | Mean | (SD) | Mean | (SD) | Effects | |
| | 0 | 5.6 | (1.9) | 5.7 | (2.1) | Group | 0.24 |
| | 60 | 5.8 | (2.0) | 6.2 | (1.9)* | Time | <0.001 |
| | 120 | 6.2 | (1.7)* | 6.3 | (2.2) | Group × | 0.33 |
| | | | | | | Time | |
| | 180 | 6.1 | (1.9)* | 6.5 | (2.0)* | | |

| | | |
|---|---|---|
| Within | 0.001 | 0.007 |
| Group (p) | | |

*= Different from 0-minute time point (p < 0.05)

TABLE 8
Changes from Baseline

| | Mean Difference ± SE | p | 95% CI | d |
|---|---|---|---|---|
| 60 min-0 min | 0.22 ± 0.22 | 0.34 | (−0.24, 0.67) | 0.27 |
| 120 min-0 min | −0.11 ± 0.25 | 0.66 | (−0.64, 0.41) | −0.11 |
| 180 min-0 min | 0.24 ± 0.23 | 0.31 | (−0.24, 0.73) | 0.24 |

Notably, the group×sex×time interaction tended to be different (p=0.034) for VAS Sustained Energy ratings. This means that the two sexes responded differently between the two supplemental conditions across time. To effectively decompose this interaction, separate 2×4 (group×time) mixed factorial ANOVA with repeated measures on time were completed separately for each gender. For males, no significant group×time interaction was observed for the males (p=0.68), however unexpectedly, for females a significant group×time interaction was observed for the females (p=0.022). Table 9 shows further data depicting mean differences.

TABLE 9
Mean differences

| | | | | Mean | | | |
|---|---|---|---|---|---|---|---|
| | Group | Mean | (SD) | Difference ± SE | p | 95% CI | d |
| Δ @ 60 min | A | 0.12 | (0.87) | 0.74 ± 0.29 | 0.03 | (0.10, 1.38) | 0.86 |
| | B | 0.85 | (0.82) | | | | |
| Δ @ 120 min | A | 0.85 | (0.71) | 0.02 ± 0.35 | 0.95 | (−0.75, 0.79) | 0.03 |
| | B | 0.88 | (1.50) | | | | |
| Δ @ 180 min | A | 0.55 | (0.82) | 0.72 ± 0.31 | 0.04 | (0.04, 1.41) | 0.62 |
| | B | 1.28 | (1.45) | | | | |

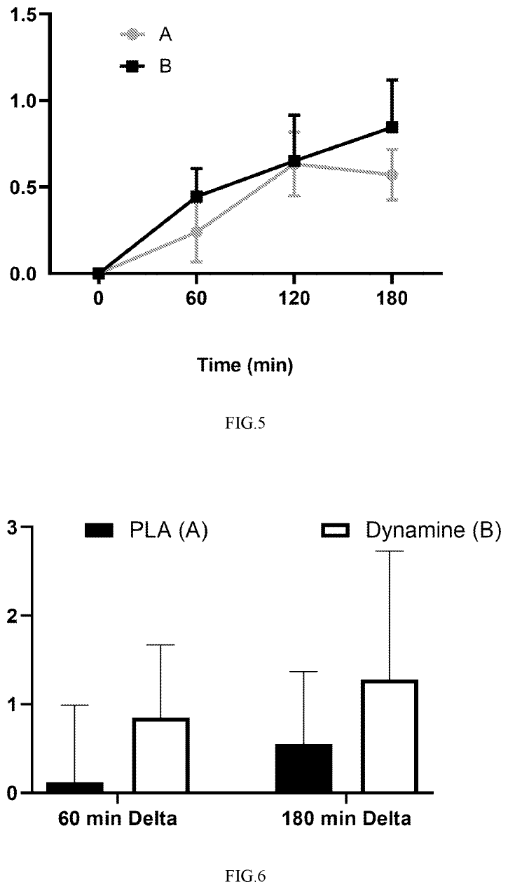

Based on the above, it can therefore clearly be seen that for Day 0 vs. Day 4 no significant group×time interaction (p=0.38) existed as is also graphically shown in FIG. 4. Similarly, at Day 4 (0-minute vs. 60, 120, and 180 minutes) no significant group×time interaction (p=0.33) was observed as is also graphically shown in FIG. 5. Changes from baseline are also shown in FIG. 6. Unexpectedly, a 3-way gender×condition×time interaction was present (p=0.034). here, for males no significant group×time interaction was observed, while for females a significant group×time interaction was observed (p=0.020). Moreover, and particularly in view of the timing of the observed response, it should be recognized that an increase in sustained energy is even more surprising than an increase in acute energy.

VAS-Mental Stamina: In this subjective test, subjects were asked to rate their personal level of energy on a VAS scale using the prompt of “My level of mental stamina right now is . . . ” (with rating lowest possible to highest possible)”. Results were collected as noted above and Table 10 provides exemplary results for Day 0 versus Day 4 ratings, whereas Table 11 provides exemplary acute results measured at Day 4 over a duration of 180 minutes. Table 12 shows changes from baseline.

TABLE 10
Day 0 vs Day 4

| | | Group A | | Group B | | Between-Group | |
|---|---|---|---|---|---|---|---|
| Variable | | Mean | (SD) | Mean | (SD) | Effects | |
| | Day 0 | 5.8 | (1.8) | 5.7 | (1.9) | Group | 0.32 |
| | Day 4 | 5.6 | (2.0) | 6.0 | (1.9) | Time | 0.74 |
| Within | | 0.27 | | 0.13 | | Group × Time | 0.11 |
| Group (p) | | | | | | | |

TABLE 11
Day 4: 0, 60, 120, and 180 minutes

| | | A | | B | | Between-Group | |
|---|---|---|---|---|---|---|---|
| Variable | Time | Mean | (SD) | Mean | (SD) | Effects | |
| | 0 | 5.6 | (2.0) | 6.0 | (1.9) | Group | 0.15 |
| | 60 | 6.0 | (1.9) | 6.3 | (1.9) | Time | 0.003 |
| | 120 | 6.1 | (1.7)* | 6.3 | (2.1) | Group × Time | 0.41 |
| | 180 | 6.0 | (1.9)* | 6.6 | (1.9) | | |
| Within | | 0.007 | | 0.04 | | | |
| Group (p) | | | | | | | |

*= Different from 0-minute time point (p < 0.05)

TABLE 12
Changes from Baseline

| | Mean Difference ± SE | p | 95% CI | d |
|---|---|---|---|---|
| 60 min-0 min | −0.12 ± 0.22 | 0.60 | (−0.56, 0.33) | −0.15 |
| 120 min-0 min | −0.21 ± 0.28 | 0.46 | (−0.80, 0.37) | −0.21 |
| 180 min-0 min | 0.16 ± 0.24 | 0.52 | (−.34, 0.65) | 0.15 |

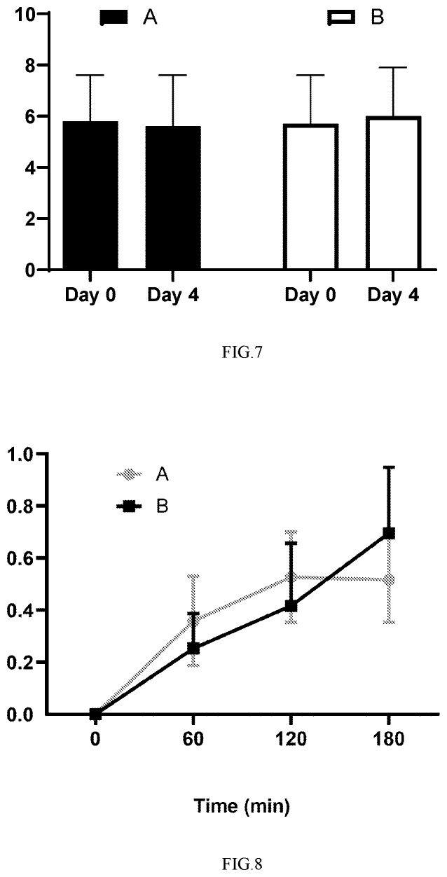

Based on the above, it can therefore clearly be seen that for Day 0 vs. Day 4 no significant group×time interaction (p=0.11) existed as is also graphically shown in FIG. 7. Similarly, at Day 4 (0-minute vs. 60, 120, and 180 minutes) no significant group×time interaction (p=0.41) was observed as is also graphically shown in FIG. 8. However, here as well, it was unexpectedly observed that mental stamina increased at 180 minutes, which is particularly surprising given the hour half-life of methylliberine.

VAS-Ability to focus: In this subjective test, subjects were asked to rate their personal level of energy on a VAS scale using the prompt of “My ability to focus right now is . . . ” (with rating lowest possible to highest possible)”. Results were collected as noted above and Table 13 provides exemplary results for Day 0 versus Day 4 ratings, whereas Table 14 provides exemplary acute results measured at Day 4 over a duration of 180 minutes. Table 15 shows changes from baseline.

TABLE 13
Day 0 vs Day 4

| | | Group A | | Group B | | Between-Group | |
|---|---|---|---|---|---|---|---|
| Variable | | Mean | (SD) | Mean | (SD) | Effects | |
| | Day 0 | 5.9 | (1.9) | 5.9 | (1.8) | Group | 0.61 |
| | Day 4 | 6.0 | (1.9) | 6.0 | (2.0) | Time | 0.58 |
| Within | | 0.59 | | 0.77 | | Group × Time | 0.91 |
| Group (p) | | | | | | | |

TABLE 14
Day 4: 0, 60, 120, and 180 minutes

| | | A | | B | | Between-Group | |
|---|---|---|---|---|---|---|---|
| | Time | Mean | (SD) | Mean | (SD) | Effects | |
| | 0 | 6.0 | (1.9) | 6.0 | (2.0) | Group | 0.18 |
| | 60 | 6.0 | (2.0) | 6.4 | (1.8) | Time | 0.03 |
| | 120 | 6.2 | (1.8) | 6.5 | (2.0) | Group × Time | 0.33 |
| | 180 | 6.2 | (1.9) | 6.7 | (2.0) | | |
| Within | | 0.47 | | 0.04 | | | |
| Group (p) | | | | | | | |

TABLE 15
Changes from Baseline

| | Mean Difference ± SE | p | 95% CI | d |
|---|---|---|---|---|
| 60 min-0 min | 0.35 ± 0.30 | 0.25 | (−0.26, 0.96) | 0.34 |
| 120 min-0 min | 0.20 ± 0.26 | 0.45 | (−0.34, 0.74) | 0.20 |
| 180 min-0 min | 0.44 ± 0.24 | 0.08 | (−0.06, 0.93) | 0.38 |

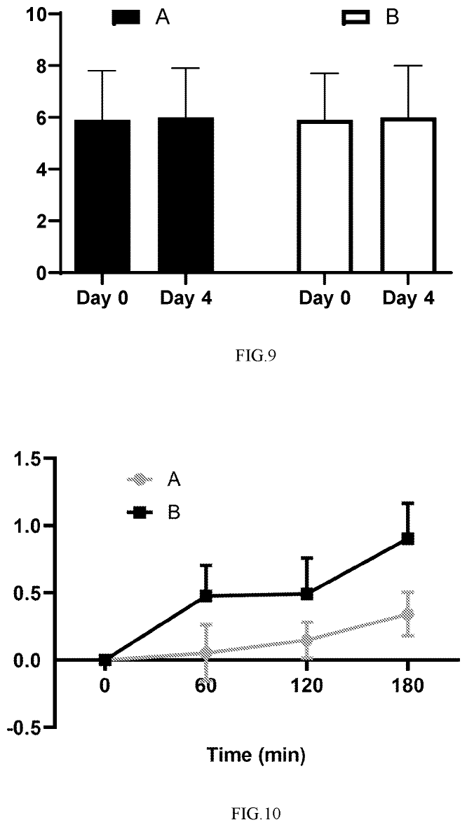

Based on the above, it can therefore clearly be seen that for Day 0 vs. Day 4 no significant group×time interaction (p=0.91) existed as is also graphically shown in FIG. 9. Similarly, at Day 4 (0-minute vs. 60, 120, and 180 minutes) no significant group×time interaction (p=0.33) was observed as is also graphically shown in FIG. 10. However, it was again unexpectedly observed that a positive impact was realized at 180 minutes. In this context it is noted that “focus” is often a result of positive impacts on brainwaves. Previous results have shown a positive brainwave activation (decrease in alpha waves; increase in theta (and delta)) with a triple-combination of methylliberine, theacrine and caffeine. So, methylliberine is also believed to impact brain waves positively. Once more, it was surprising to observe a lasting impact on cognitive function well past methylliberine's half-life.

VAS-Ability to concentrate: In this subjective test, subjects were asked to rate their personal level of energy on a VAS scale using the prompt of “My ability to concentrate right now is . . . ” (with rating lowest possible to highest possible)”. Results were collected as noted above and Table 16 provides exemplary results for Day 0 versus Day 4 ratings, whereas Table 17 provides exemplary acute results measured at Day 4 over a duration of 180 minutes. Table 18 shows changes from baseline.

TABLE 16
Day 0 vs. Day 4

| | | Group A | | Group B | | Between-Group | |
|---|---|---|---|---|---|---|---|
| Variable | | Mean | (SD) | Mean | (SD) | Effects | |
| | Day 0 | 5.9 | (2.0) | 5.9 | (1.8) | Group | 0.89 |
| | Day 4 | 5.9 | (1.8) | 5.9 | (2.0) | Time | 0.91 |
| Within | | 0.91 | | 0.96 | | Group × Time | 0.98 |
| Group (p) | | | | | | | |

TABLE 17
Day 4: 0, 60, 120, and 180 minutes

| | | A | | B | | Between-Group | |
|---|---|---|---|---|---|---|---|
| Variable | Time | Mean | (SD) | Mean | (SD) | Effects | |
| | 0 | 5.9 | (1.8) | 5.9 | (2.0) | Group | 0.17 |
| | 60 | 6.3 | (1.9) | 6.5 | (1.7)* | Time | 0.002 |
| | 120 | 6.3 | (1.7) | 6.5 | (2.0) | Group × Time | 0.03 |
| | 180 | 6.1 | (1.8) | 6.8 | (1.8)* | | |
| Within | | 0.16 | | 0.002 | | | |
| Group (p) | | | | | | | |

*= Different from 0-minute time point (p < 0.05)
† = Between-group difference at designated time point (p < 0.05)

TABLE 18
Changes from Baseline

| | Mean Difference ± SE | p | 95% CI | d |
|---|---|---|---|---|
| 60 min-0 min | 0.26 ± 0.26 | 0.34 | (−0.29, 0.80) | 0.28 |
| 120 min-0 min | 0.15 ± 0.27 | 0.59 | (−0.41, 0.70) | 0.14 |
| 180 min-0 min | 0.70 ± 0.23 | 0.006 | (0.22, 1.18) | 0.68 |

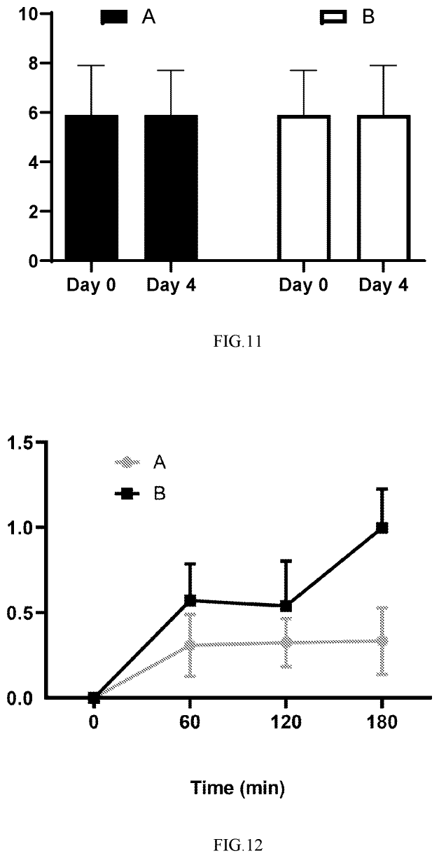

Based on the above, it can therefore clearly be seen that for Day 0 vs. Day 4 no significant group×time interaction (p=0.98) existed as is also graphically shown in FIG. 11. However unexpectedly, at Day 4 (0-minute vs. 60, 120, and 180 minutes) a significant group×time interaction was present (p=0.03) as is graphically shown in FIG. 12. Moreover, it should be noted that the VAS ratings in group B (Active) increased to a remarkably greater degree than group A (placebo) at the 180-minute time point (p=0.006).

VAS-Motivation: In this subjective test, subjects were asked to rate their personal level of energy on a VAS scale using the prompt of “My motivation to accomplish difficult tasks right now is . . . ” (with rating lowest possible to highest possible)”. Results were collected as noted above and Table 19 provides exemplary results for Day 0 versus Day 4 ratings, whereas Table 20 provides exemplary acute results measured at Day 4 over a duration of 180 minutes. Table 21 shows changes from baseline.

TABLE 19
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 5.7 | (2.0) | 5.8 | (2.0) | Group | 0.45 |
| | Day 4 | 5.5 | (2.0) | 5.7 | (1.9) | Time | 0.29 |

| | | | | |
|---|---|---|---|---|
| Within Group (p) | 0.20 | 0.79 | Group × Time | 0.63 |

TABLE 20
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 5.5 | (2.0) | 5.7 | (1.9) | Group | 0.19 |
| | 60 | 6.1 | (1.9)* | 6.2 | (1.8) | Time | <0.001 |
| | 120 | 6.2 | (1.9)* | 6.2 | (2.0) | Group × Time | 0.02 |
| | 180 | 5.8 | (2.0) | 6.6 | (1.8)* | | |

| | | |
|---|---|---|
| Within Group (p) | 0.003 | 0.001 |

*= Different from 0-minute time point (p < 0.05)

TABLE 21
Changes from Baseline

| | Mean Difference ± SE | p | 95% CI | d |
|---|---|---|---|---|
| 60 min-0 min | −0.07 ± 0.27 | 0.79 | (−0.63, 0.49) | −0.08 |
| 120 min-0 min | −0.25 ± 0.27 | 0.35 | (−0.80, 0.30) | −0.25 |
| 180 min-0 min | 0.53 ± 0.27 | 0.06 | (−0.02, 1.08) | 0.50 |

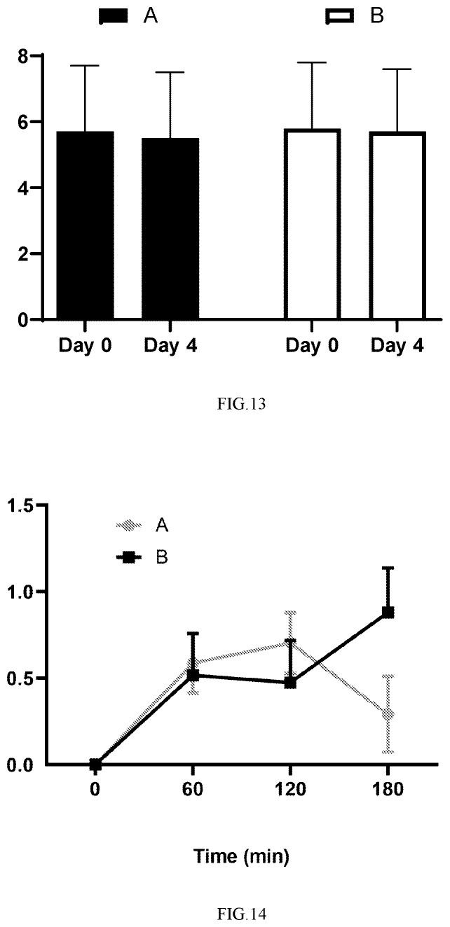

Based on the above, it can therefore clearly be seen that for Day 0 vs. Day 4 no significant group×time interaction (p=0.63) existed as is also graphically shown in FIG. 13. Remarkably, at Day 4 (0-minute vs. 60, 120, and 180 minutes) a significant group×time interaction was present (p=0.02) as is graphically shown in FIG. 14. Additionally, it is noted that a trend (p=0.06) was noted for a greater improvement in group B (Active) vs. group A (placebo) at the 180-minute time point. Of note, it should be appreciated that motivation is often a function of dopamine activation. As such, is it contemplated that methylliberine may also have an agonist effect on dopamine receptors or signaling.

VAS-Drive: In this subjective test, subjects were asked to rate their personal level of energy on a VAS scale using the prompt of “My drive right now is . . . ” (with rating lowest possible to highest possible)”. Results were collected as noted above and Table 22 provides exemplary results for Day 0 versus Day 4 ratings, whereas Table 23 provides exemplary acute results measured at Day 4 over a duration of 180 minutes. Table 24 shows changes from baseline.

TABLE 22
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 5.7 | (1.9) | 5.8 | (1.9) | Group | 0.37 |
| | Day 4 | 5.7 | (2.0) | 5.9 | (1.9) | Time | 0.65 |

| | | | | |
|---|---|---|---|---|
| Within Group (p) | 0.77 | 0.78 | Group × Time | 0.97 |

TABLE 23
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 5.7 | (2.0) | 5.9 | (1.9) | Group | 0.45 |
| | 60 | 6.0 | (1.9) | 6.1 | (2.1) | Time | 0.02 |
| | 120 | 6.3 | (1.7)* | 6.2 | (2.0) | Group × Time | 0.36 |
| | 180 | 6.0 | (1.8) | 6.4 | (2.1) | | |

| | | |
|---|---|---|
| Within Group (p) | 0.05 | 0.07 |

*= Different from 0-minute time point (p < 0.05)

TABLE 24
Changes from Baseline

| | Mean Difference ± SE | p | 95% CI | d |
|---|---|---|---|---|
| 60 min-0 min | −0.06 ± 0.27 | 0.82 | (−0.63, 0.50) | −0.07 |
| 120 min-0 min | −0.16 ± 0.28 | 0.58 | (−0.73, 0.42) | −0.15 |
| 180 min-0 min | 0.33 ± 0.35 | 0.35 | (−0.39, 1.04) | 0.26 |

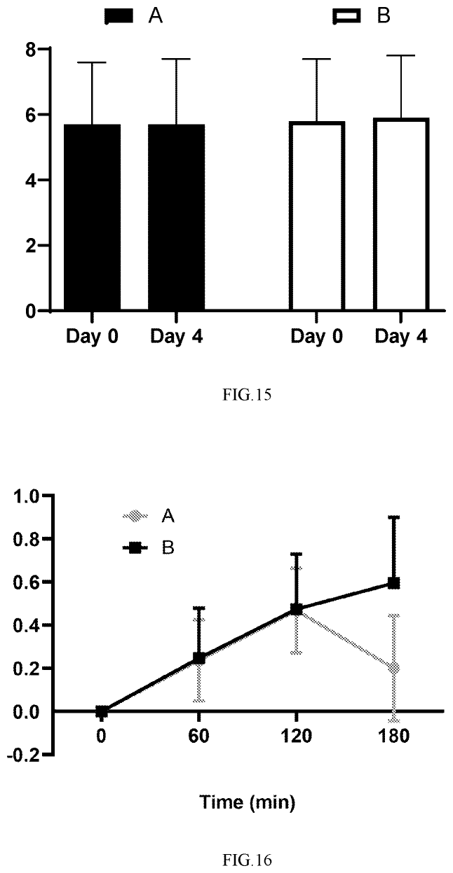

Based on the above, it can therefore clearly be seen that for Day 0 vs. Day 4 no significant group×time interaction (p=0.97) existed as is also graphically shown in FIG. 15. Similarly, at Day 4 (0-minute vs. 60, 120, and 180 minutes) no significant group×time interaction (p=0.23) was observed as is also graphically shown in FIG. 16.

VAS-Vigor: In this subjective test, subjects were asked to rate their personal level of energy on a VAS scale using the prompt of “My level of vigor right now is . . . ” (with rating lowest possible to highest possible)”. Results were collected as noted above and Table 25 provides exemplary results for Day 0 versus Day 4 ratings, whereas Table 26 provides exemplary acute results measured at Day 4 over a duration of 180 minutes. Table 27 shows changes from baseline. Of note, it should be appreciated that drive is also often a function of dopamine activation. As above, is it therefore contemplated that methylliberine may also have an agonist effect on dopamine receptors or signaling.

TABLE 25
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 5.8 | (1.8) | 5.8 | (2.0) | Group | 0.83 |
| | Day 4 | 5.7 | (2.0) | 5.8 | (2.0) | Time | 0.97 |

| | | | | |
|---|---|---|---|---|
| Within Group (p) | 0.81 | 0.85 | Group × Time | 0.78 |

TABLE 26
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 5.7 | (2.0) | 5.8 | (2.0) | Group | 0.47 |
| | 60 | 6.1 | (2.0) | 6.2 | (2.0) | Time | 0.002 |
| | 120 | 6.3 | (1.8)* | 6.3 | (1.9) | Group × Time | 0.10 |
| | 180 | 5.9 | (1.8) | 6.4 | (2.0)* | | |

| | | |
|---|---|---|
| Within Group (p) | 0.009 | 0.009 |

*= Different from 0-minute time point (p < 0.05)

TABLE 27
Changes from Baseline

| | Mean Difference ± SE | p | 95% CI | d |
|---|---|---|---|---|
| 60 min-0 min | 0.02 ± 0.23 | 0.94 | (−0.45, 0.48) | 0.02 |
| 120 min-0 min | −0.12 ± 0.25 | 0.64 | (−0.64, 0.40) | 0.13 |
| 180 min-0 min | 0.43 ± 0.28 | 0.13 | (−0.14, 1.01) | −0.41 |

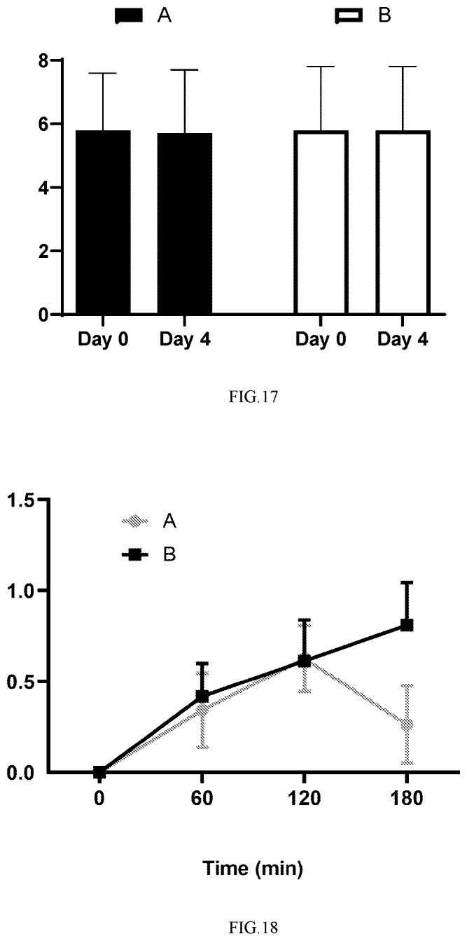

Based on the above, it can therefore clearly be seen that for Day 0 vs. Day 4 no significant group×time interaction (p=0.78) existed as is also graphically shown in FIG. 17. Similarly, at Day 4 (0-minute vs. 60, 120, and 180 minutes) no significant group×time interaction (p=0.23) was observed as is also graphically shown in FIG. 18.

VAS-Positivity: In this subjective test, subjects were asked to rate their personal level of energy on a VAS scale using the prompt of “My level of positive outlook right now is . . . ” (with rating lowest possible to highest possible)”. Results were collected as noted above and Table 28 provides exemplary results for Day 0 versus Day 4 ratings, whereas Table 29 provides exemplary acute results measured at Day 4 over a duration of 180 minutes. Table 30 shows changes from baseline.

TABLE 28
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 6.8 | (1.7) | 6.6 | (1.9)* | Group | 0.64 |
| | Day 4 | 6.4 | (1.9) | 6.4 | (2.0) | Time | 0.01 |

| | | | | |
|---|---|---|---|---|
| Within Group (p) | 0.04 | 0.24 | Group × Time | 0.62 |

*= Different from Day 0 (p < 0.05).

TABLE 29
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 6.4 | (1.9) | 6.4 | (2.0) | Group | 0.62 |
| | 60 | 6.7 | (1.8) | 6.8 | (1.9) | Time | <0.001 |
| | 120 | 7.0 | (1.7)* | 7.0 | (1.7) | Group × Time | 0.08 |
| | 180 | 6.8 | (1.9) | 7.0 | (1.7)* | | |

| | | |
|---|---|---|
| Within Group (p) | <0.001 | 0.002 |

*= Different from 0-minute time point (p < 0.05)

TABLE 30
Changes from Baseline

| | Mean Difference ± SE | p | 95% CI | d |
|---|---|---|---|---|
| 60 min-0 min | 0.07 ± 0.19 | 0.73 | (−0.33, 0.46) | 0.08 |
| 120 min-0 min | −0.05 ± 0.25 | 0.85 | (−0.56, 0.46) | 0.05 |
| 180 min-0 min | 0.46 ± 0.27 | 0.10 | (−0.09, 1.01) | 0.43 |

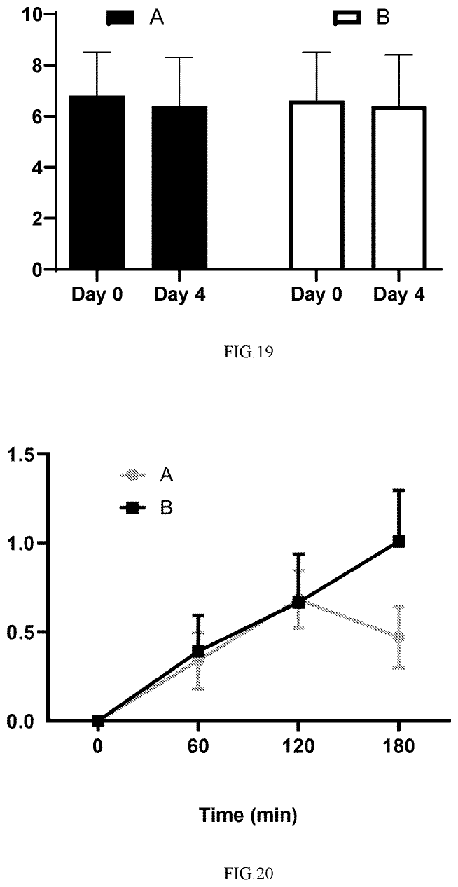

Based on the above, it can therefore clearly be seen that for Day 0 vs. Day 4 no significant group×time interaction (p=0.62) existed as is also graphically shown in FIG. 19. Similarly, at Day 4 (0-minute vs. 60, 120, and 180 minutes) no significant group×time interaction (p=0.08) was observed as is also graphically shown in FIG. 20. Notably, the 3-way gender×condition×time interaction tended to be significant (p=0.064). Here, for males no significant group×time interaction was observed for (p=0.68), whereas for females a significant group×time interaction was observed for female (p=0.022).

With regard to this unexpected result and the other results in which the response appeared to be gender-associated, the inventor contemplates that there is a dose response by body weight of the subjects. Indeed, based on the data above (and other considerations not shown here) methylliberine appeared to be most effectively dosed at on a mg per kg of body weight. Thus, women appeared to have with at least some selected metrics a more robust response as their bodyweight was lower than that of men. It should also be noted that positive responses to caffeine declined after two hours, with a typical a decrease in mood, concentration, energy, motivation and cognitive performance metrics, despite caffeine's half-life being longer than that of methylliberine. Such findings were entirely unexpected.

VAS-Mood: In this subjective test, subjects were asked to rate their personal level of energy on a VAS scale using the prompt of “My mood right now is . . . ” (with rating lowest possible to highest possible)”. Results were collected as noted above and Table 31 provides exemplary results for Day 0 versus Day 4 ratings, whereas Table 32 provides exemplary acute results measured at Day 4 over a duration of 180 minutes. Table 33 shows changes from baseline.

TABLE 31
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 6.6 | (1.8) | 6.5 | (1.9) | Group | 0.54 |
| | Day 4 | 6.2 | (2.0) | 6.1 | (2.0) | Time | 0.01 |

| | | | | |
|---|---|---|---|---|
| Within Group (p) | 0.18 | 0.08 | Group × Time | 0.70 |

TABLE 32
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 6.2 | (2.0) | 6.1 | (2.0) | Group | 0.48 |
| | 60 | 6.5 | (1.9) | 6.7 | (1.8)* | Time | <0.001 |
| | 120 | 6.8 | (1.7)* | 7.0 | (1.7)* | Group × Time | 0.04 |
| | 180 | 6.6 | (1.7) | 7.1 | (1.7)* | | |

| | | |
|---|---|---|
| Within Group (p) | 0.009 | <0.001 |

*= Different from 0-minute time point (p < 0.05)
† = Between-group difference at designated time point (p < 0.05)

TABLE 33
Changes from Baseline

| | Mean Difference ± SE | p | 95% CI | d |
|---|---|---|---|---|
| 60 min-0 min | 0.38 ± 0.18 | 0.05 | (−0.01, 0.76) | 0.43 |
| 120 min-0 min | 0.45 ± 0.27 | 0.11 | (−0.11, 1.02) | 0.41 |
| 180 min-0 min | 0.72 ± 0.30 | 0.03 | (1.35, 2.37) | 0.65 |

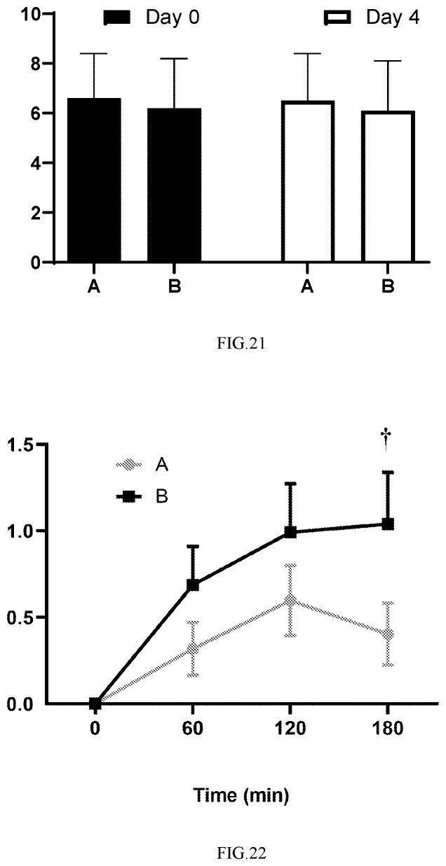

Based on the above, it can therefore clearly be seen that for Day 0 vs. Day 4 no significant group×time interaction (p=0.70) existed as is also graphically shown in FIG. 21. However unexpectedly, at Day 4 (0-minute vs. 60, 120, and 180 minutes) a significant group×time interaction was present (p=0.04) as is graphically shown in FIG. 22. Moreover, it should be noted that the VAS ratings in group B (Active) unexpectedly increased to a greater degree than group A (placebo) at the 60-minute (p=0.05) and 180-minute time points (p=0.03). Such finding is once more surprising given the Cₘₐₓ of methylliberine is 0.6-0.9 hours, and its half-life is 1-1.4 hours.

VAS-Well-Being: In this subjective test, subjects were asked to rate their personal level of energy on a VAS scale using the prompt of “My feeling of well-being right now is . . . ” (with rating lowest possible to highest possible)”. Results were collected as noted above and Table 34 provides exemplary results for Day 0 versus Day 4 ratings, whereas Table 35 provides exemplary acute results measured at Day 4 over a duration of 180 minutes. Table 36 shows changes from baseline.

TABLE 34
Day 0 vs. Day 4

| | | Group A | | Group b | | | |
|---|---|---|---|---|---|---|---|
| Variable | | Mean | (SD) | Mean | (SD) | Between-Group Effects | |
| | Day 0 | 7.0 | (1.7) | 6.8 | (2.0) | Group | 0.15 |
| | Day 4 | 6.6 | (1.8) | 6.4 | (1.7) | Time | 0.01 |

| | | | | |
|---|---|---|---|---|
| Within Group (p) | 0.06 | 0.07 | Group × Time | 0.92 |

TABLE 35
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 6.6 | (1.8) | 6.4 | (1.7) | Group | 0.75 |
| | 60 | 6.9 | (1.8) | 6.8 | (1.7) | Time | <0.001 |
| | 120 | 7.0 | (1.6) | 7.3 | (1.6)* | Group × Time | 0.09 |
| | 180 | 6.9 | (1.8) | 7.2 | (1.6)* | | |

| | | |
|---|---|---|
| Within Group (p) | 0.06 | 0.001 |

*= Different from 0-minute time point (p < 0.05)
† = Between-group difference at designated time point (p ≤ 0.05)

TABLE 36
Changes from Baseline

| | Mean Difference ± SE | p | 95% CI | d |
|---|---|---|---|---|
| 60 min-0 min | 0.16 ± 0.16 | 0.34 | (−0.17, 0.48) | 0.22 |
| 120 min-0 min | 0.46 ± 0.29 | 0.12 | (−0.13, 1.05) | 0.45 |
| 180 min-0 min | 0.54 ± 1.34 | 0.05 | (−0.01, 1.10) | 0.52 |

Post-hoc power calculation with the observed effect size (d=0.52) at 180 minutes indicated that a total sample size of 25 subjects were needed to achieve a power (1-B) of 0.80 at an alpha level of 0.05.

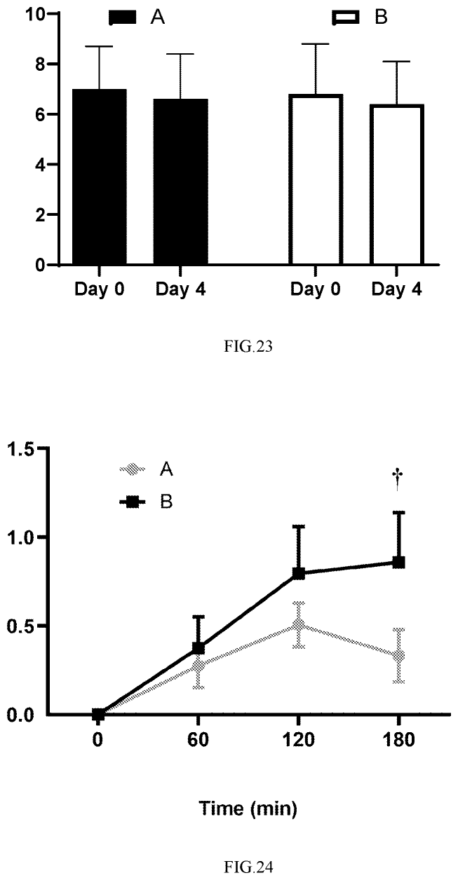

Based on the above, it can therefore clearly be seen that for Day 0 vs. Day 4 no significant group×time interaction (p=0.92) existed as is also graphically shown in FIG. 23. However, at Day 4 (0-minute vs. 60, 120, and 180 minutes) the group×time interaction (p=0.23) tended to be different as is also graphically shown in FIG. 24. Once more, the VAS ratings in group B (Active) increased to a greater degree than group A (placebo) at the 180-minute time point (p=0.05), which is an unexpected finding in light of the relatively short half-life time of methylliberine as already discussed above.

VAS-Ability to tolerate stress: In this subjective test, subjects were asked to rate their personal level of energy on a VAS scale using the prompt of “My ability to tolerate stress right now is . . . ” (with rating lowest possible to highest possible)”. Results were collected as noted above and Table 37 provides exemplary results for Day 0 versus Day 4 ratings, whereas Table 38 provides exemplary acute results measured at Day 4 over a duration of 180 minutes. Table 39 shows changes from baseline.

TABLE 37
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 6.1 | (1.9) | 6.1 | (2.0) | Group | 0.89 |
| | Day 4 | 5.8 | (2.1) | 5.7 | (2.0) | Time | 0.002 |

| | | | | |
|---|---|---|---|---|
| Within Group (p) | 0.06 | 0.12 | Group × Time | 0.90 |

TABLE 38
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 5.8 | (2.1) | 5.7 | (2.0) | Group | 0.29 |
| | 60 | 6.0 | (2.0) | 6.2 | (2.1) | Time | 0.03 |
| | 120 | 6.1 | (2.0) | 6.4 | (1.9)* | Group × Time | 0.08 |
| | 180 | 5.8 | (2.0) | 6.4 | (1.9) | | |

| | | |
|---|---|---|
| Within Group (p) | 0.21 | 0.01 |

*= Different from 0-minute time point (p < 0.05)
† = Between-group difference at designated time point (p < 0.05)

TABLE 39
Changes from Baseline

| | Mean Difference ± SE | p | 95% CI | d |
|---|---|---|---|---|
| 60 min-0 min | 0.17 ± 0.18 | 0.35 | (−0.20, 0.54) | 0.21 |
| 120 min-0 min | 0.28 ± 1.42 | 0.34 | (−0.31, 0.86) | 0.27 |
| 180 min-0 min | 0.67 ± 0.30 | 0.04 | (0.04, 1.29) | 0.54 |

Post-hoc power calculation with the observed effect size (d=0.52) at 180 minutes indicated that a total sample size of 23 subjects were needed to achieve a power (1-B) of 0.80 at an alpha level of 0.05.

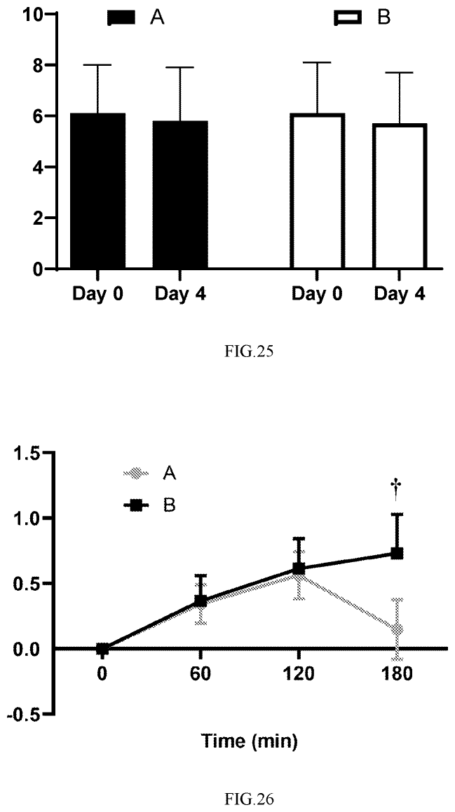

Based on the above, it can therefore clearly be seen that for Day 0 vs. Day 4 no significant group×time interaction (p=0.27) existed as is also graphically shown in FIG. 25. Notably, at Day 4 (0-minute vs. 60, 120, and 180 minutes) the group×time interaction (p=0.08) tended to be different as is graphically shown in FIG. 26. Notably, the VAS ratings in group B (Active) increased to a greater degree than group A (placebo) at the 180-minute time point (p=0.04). Such finding is once more surprising given the Cₘₐₓ of methylliberine is 0.6-0.9 hours, and the half-life is 1-1.4 hours.

Table 40 illustrates the results for analyzing the data in the VAS experiments by area under the curve.

TABLE 40
Area under the curve

| | | | | | Mean Difference ± | | |
|---|---|---|---|---|---|---|---|
| Variable | Group | Mean | (SD) | p | SE | 95% CI | d |
| Energy | A | 1065 | (328) | 0.19 | 49.6 ± 36.3 | (−25, 125) | 0.15 |
| | B | 1115 | (351) | | | | |
| Sustained | A | 1118 | (353) | 0.29 | 45.2 ± 41.7 | (−41, 131) | −0.13 |
| | B | 1073 | (321) | | | | |
| Mental | A | 1074 | (324) | 0.21 | 59.2 ± 46.2 | (−36, 155) | 0.18 |
| | B | 1133 | (343) | | | | |
| Focus | A | 1100 | (326) | 0.19 | 55.3 ± 40.9 | (−29, 140) | 0.17 |
| | B | 1155 | (336) | | | | |
| Concentrate | A | 1113 | (310) | 0.23 | 48.1 ± 38.7 | (−32, 128) | 0.16 |
| | B | 1162 | (319) | | | | |
| Motivation | A | 1077 | (331) | 0.33 | 36.8 ± 37.4 | (−40, 114) | 0.11 |
| | B | 1113 | (324) | | | | |
| Drive | A | 1089 | (330) | 0.59 | 20.4 ± 37.8 | (−58, 98) | 0.06 |
| | B | 1110 | (353) | | | | |
| Vigor | A | 1090 | (330) | 0.60 | 21.8 ± 41.5 | (−64, 108) | 0.07 |
| | B | 1112 | (343) | | | | |
| Positivity | A | 1220 | (313) | 0.73 | 15.0 ± 43.0 | (−74, 104) | 0.05 |
| | B | 1235 | (317) | | | | |
| Mood | A | 1182 | (318) | 0.46 | 37.4 ± 250.7 | (−66, 141) | 0.12 |
| | B | 1220 | (305) | | | | |
| Well-Being | A | 1254 | (280) | 0.73 | 14.9 ± 43.0 | (−74, 104) | −0.05 |
| | B | 1239 | (300) | | | | |
| Ability | A | 1120 | (341) | 0.33 | 45.5 ± 45.6 | (−49, 140) | −0.13 |
| | B | 1075 | (346) | | | | |

For the experiments conducted above, no significant changes were observed when analyzed by an area under the curve perspective.

### Stroop Test

The Stroop Color and Word Test (SCWT, or Stroop test) is a neuropsychological test extensively used to assess the ability to inhibit cognitive interference that occurs when the processing of a specific stimulus feature impedes the simultaneous processing of a second stimulus attribute, well-known as the Stroop effect (see e.g., Front Psychol. 2017; 8:557). Here, subjects were required to read three different tables as fast as possible. Two of them represent the “congruous condition” in which participants are required to read names of colors (‘color-words’) printed in black ink (W) and name different color patches (C). However, in the third table, the color-word (CW) condition, color-words are printed in an inconsistent color ink (e.g., the word “red” is printed in green ink). In this incongruent condition, participants are required to name the color of the ink instead of reading the word. In other words, the participants are required to perform a less automated task (i.e., naming ink color) while inhibiting the interference arising from a more automated task (i.e., reading the word). This difficulty in inhibiting the more automated process is called the Stroop effect. The Stroop test is predominantly used to measure the ability to inhibit cognitive interference. However, some sources also report its application to measure other cognitive functions such as attention, processing speed, cognitive flexibility, and working memory.

Stroop-Total Score: The results for this test were based on a total score comparing performance between Day 0 and Day 4, with Day 4 including tests over a duration of 180 minutes. Results were collected as noted above and Table 41 provides exemplary results for Day 0 versus Day 4 ratings, whereas Table 42 provides exemplary acute results measured at Day 4 over a duration of 180 minutes.

TABLE 41
Day 0 vs Day 4 (total score)

| | | Group A | | Group B | | | |
|---|---|---|---|---|---|---|---|
| Variable | | Mean | (SD) | Mean | (SD) | Between-Group Effects | |
| | Day 0 | 125.2 | (18.4) | 125.0 | (17.9) | Group | 0.61 |
| | Day 4 | 124.7 | (19.2) | 127.4 | (18.8) | Time | 0.44 |

| | | | | |
|---|---|---|---|---|
| Within (p) | 0.77 | 0.20 | Group × Time | 0.29 |

TABLE 42
Day 4: 0, 60, 120, and 180 minutes (total score)

| | | A | | B | | | |
|---|---|---|---|---|---|---|---|
| Variable | Time | Mean | (SD) | Mean | (SD) | Between-Group Effects | |
| | 0 | 124.7 | (19.2) | 127.4 | (18.8) | Group | 0.21 |
| | 60 | 128.5 | (17.4) | 131.5 | (17.5)* | Time | <0.001 |
| | 120 | 131.3 | (17.0)* | 132.1 | (16.3)* | Group × Time | 0.61 |
| | 180 | 131.7 | (17.7) | 134.8 | (17.7)* | | |

| | | |
|---|---|---|
| Within Group (p) | 0.008 | <0.001 |

*= Different from 0-minute time point (p < 0.05)

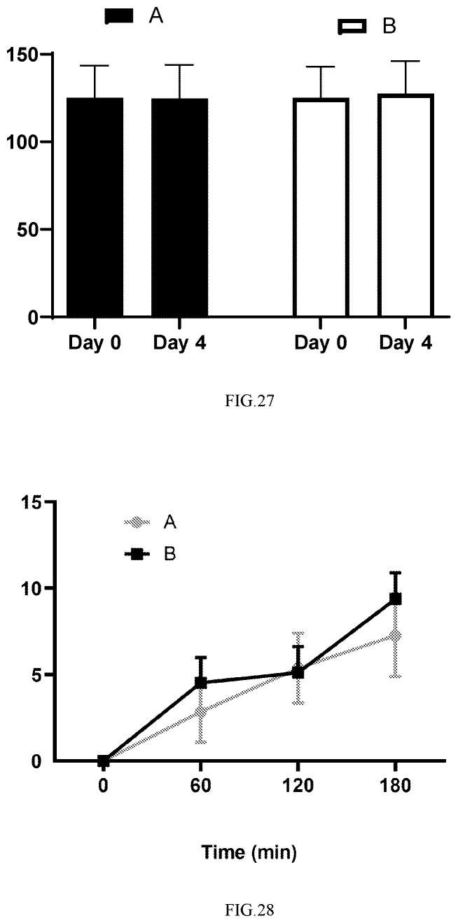

Based on the above, it can therefore clearly be seen that for Day 0 vs. Day 4 no significant group×time interaction (p=0.29) existed as is also graphically shown in FIG. 27. Similarly, at Day 4 (0-minute vs. 60, 120, and 180 minutes) no significant group×time interaction (p=0.61) was observed as is also graphically shown in FIG. 28. Unexpectedly however, when a subject's cognitive abilities were assessed over time using a Stroop and Trail Making Test (TMT), the placebo group experienced a non-significant increase (+4.4%, p=0.15) in TMT performance, whereas a group treated for 4-days with daily methylliberine supplementation significantly improved in TMT performance (+11.1%, p<0.001).

Stroop-Accuracy: The results for this test were based on an accuracy score comparing performance between Day 0 and Day 4, with Day 4 including tests over a duration of 180 minutes. Results were collected as noted above and Table 43 provides exemplary results for Day 0 versus Day 4 ratings, whereas Table 44 provides exemplary acute results measured at Day 4 over a duration of 180 minutes.

TABLE 43
Day 0 vs. Day 4 (accuracy)

| | | Group A | | Group B | | | |
|---|---|---|---|---|---|---|---|
| Variable | | Mean | (SD) | Mean | (SD) | Between-Group Effects | |
| | Day 0 | 99.1 | (1.2) | 99.2 | (1.1) | Group | 0.44 |
| | Day 4 | 99.0 | (1.1) | 99.2 | (1.3) | Time | 0.85 |

| | | | | |
|---|---|---|---|---|
| Within (p) | 0.91 | 0.86 | Group × Time | 0.99 |

TABLE 44
Day 4: 0, 60, 120, and 180 minutes (accuracy)

| | | A | | B | | | |
|---|---|---|---|---|---|---|---|
| Variable | Time | Mean | (SD) | Mean | (SD) | Between-Group Effects | |
| | 0 | 99.0 | (1.1) | 99.2 | (1.3) | Group | 0.56 |
| | 60 | 99.0 | (1.3) | 99.1 | (1.8) | Time | 0.42 |
| | 120 | 98.8 | (1.7) | 99.0 | (1.2) | Group × Time | 0.93 |
| | 180 | 98.8 | (1.3) | 98.9 | (1.8) | | |

| | | |
|---|---|---|
| Within Group (p) | 0.67 | 0.52 |

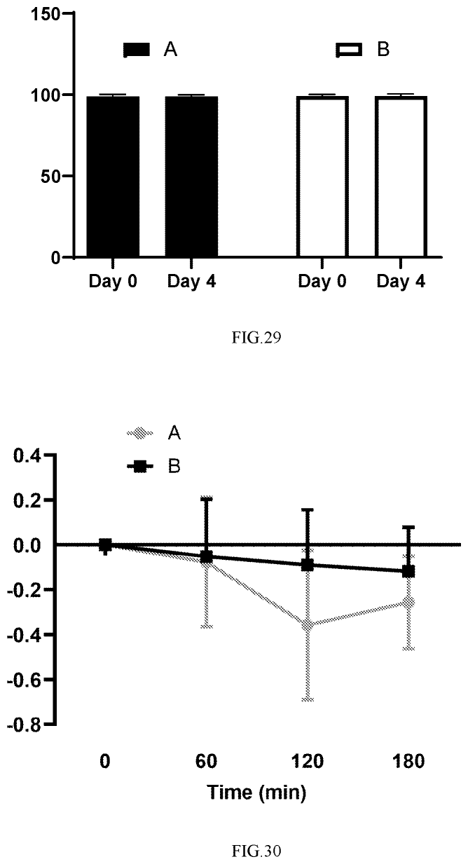

Based on the above, it can therefore clearly be seen that for Day 0 vs. Day 4 no significant group×time interaction (p=0.99) existed as is also graphically shown in FIG. 29. Similarly, at Day 4 (0-minute vs. 60, 120, and 180 minutes) no significant group×time interaction (p=0.90) was observed as is also graphically shown in FIG. 30.

Stroop-Average Time: The results for this test were based on an average time score comparing performance between Day 0 and Day 4, with Day 4 including tests over a duration of 180 minutes. Results were collected as noted above and Table 45 provides exemplary results for Day 0 versus Day 4 ratings, whereas Table 46 provides exemplary acute results measured at Day 4 over a duration of 180 minutes.

TABLE 45
Day 0 vs. Day 4 (average time)

| | | Group A | | Group B | | | |
|---|---|---|---|---|---|---|---|
| Variable | | Mean | (SD) | Mean | (SD) | Between-Group Effects | |
| | Day 0 | 0.988 | (0.14) | 0.983 | (0.13) | Group | 0.45 |
| | Day 4 | 0.989 | (0.17) | 0.961 | (0.14) | Time | 0.39 |

| | | | | |
|---|---|---|---|---|
| Within (p) | 0.97 | 0.18 | Group × Time | 0.45 |

TABLE 46
Day 4: 0, 60, 120, and 180 minutes (average time)

| | | A | | B | | | |
|---|---|---|---|---|---|---|---|
| Variable | Time | Mean | (SD) | Mean | (SD) | Between-Group Effects | |
| | 0 | 0.989 | (0.17) | 0.961 | (0.14) | Group | 0.22 |
| | 60 | 0.952 | (0.14) | 0.929 | (0.12) | Time | <0.001 |
| | 120 | 0.927 | (0.12) | 0.921 | (0.11)* | Group × Time | 0.63 |
| | 180 | 0.924 | (0.12)* | 0.908 | (0.11)* | | |

| | | |
|---|---|---|
| Within Group (p) | 0.01 | <0.001 |

*= Different from 0-minute time point (p < 0.05)

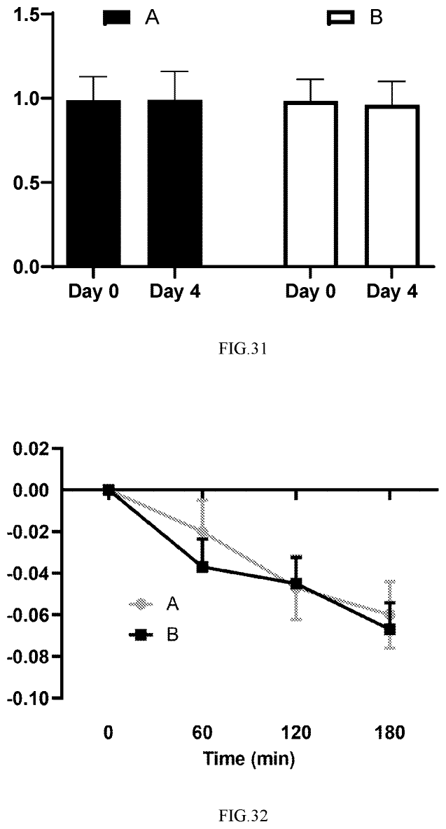

Based on the above, it can therefore clearly be seen that for Day 0 vs. Day 4 no significant group×time interaction (p=0.45) existed as is also graphically shown in FIG. 31. Similarly, at Day 4 (0-minute vs. 60, 120, and 180 minutes) no significant group×time interaction (p=0.63) was observed as is also graphically shown in FIG. 32.

### Trail Making Test

The trail making test has at least two test conditions. In Condition A, a subject is asked to draw lines to connect circled numbers in a numerical sequence (e.g., 1-2-3, etc.) as rapidly in possible. In Condition B the subject is asked to draw lines to connect circled numbers and letters in an alternating numeric and alphabetic sequence (i.e., 1-A-2-B, etc.) as rapidly as possible. Although trail making tests are conceptually very simple, they are thought to reflect a wide variety of cognitive processes including attention, visual search and scanning, sequencing and shifting, psychomotor speed, abstraction, flexibility, ability to execute and modify a plan of action, and ability to maintain two trains of thought simultaneously.

TABLE 47
Day 0 vs. Day 4

| | | Group A | | Group B | | | |
|---|---|---|---|---|---|---|---|
| Variable | | Mean | (SD) | Mean | (SD) | Between-Group Effects | |
| | Day 0 | 22.8 | (6.1) | 24.3 | (7.2) | Group | 0.56 |
| | Day 4 | 21.8 | (5.5) | 21.6 | (5.8)* | Time | 0.001 |

| | | | | |
|---|---|---|---|---|
| Within (p) | 0.15 | <0.001 | Group × Time | 0.06 |

*= Different from Day 0 (p < 0.05)

TABLE 48
Day 4: 0, 60, 120, and 180 minutes

| | | A | | B | | | |
|---|---|---|---|---|---|---|---|
| Variable | Time | Mean | (SD) | Mean | (SD) | Between-Group Effects | |
| | 0 | 21.8 | (5.5) | 21.6 | (5.8) | Group | 0.65 |
| | 60 | 21.7 | (6.0) | 20.4 | (5.2) | Time | 0.009 |
| | 120 | 21.1 | (5.4) | 20.2 | (4.1) | Group × Time | 0.13 |
| | 180 | 19.3 | (4.0)* | 20.3 | (3.8) | | |

| | | |
|---|---|---|
| Within Group (p) | 0.006 | 0.20 |

*= Different from 0-minute time point (p < 0.05)

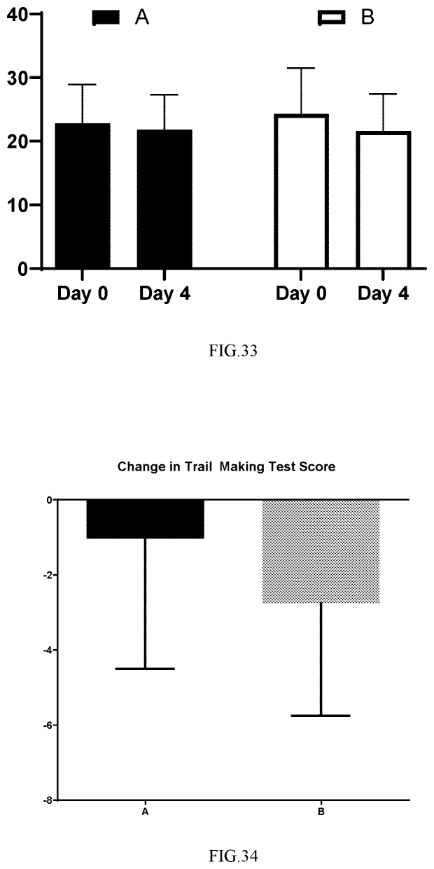

Based on the above, it can therefore clearly be seen that for Day 0 vs. Day 4 there was a trend for a group×time interaction (p=0.06) as is graphically shown in FIG. 33. Notably, Group B (Active) had significantly lower (i.e. faster) scores at day 4 than day 0 (p<0.001) changes in score as also shown in FIG. 34. However, at Day 4 (0-minute vs. 60, 120, and 180 minutes) no significant group×time interaction (p=0.61) was observed as is graphically shown in FIG. 34.

### Hemodynamics

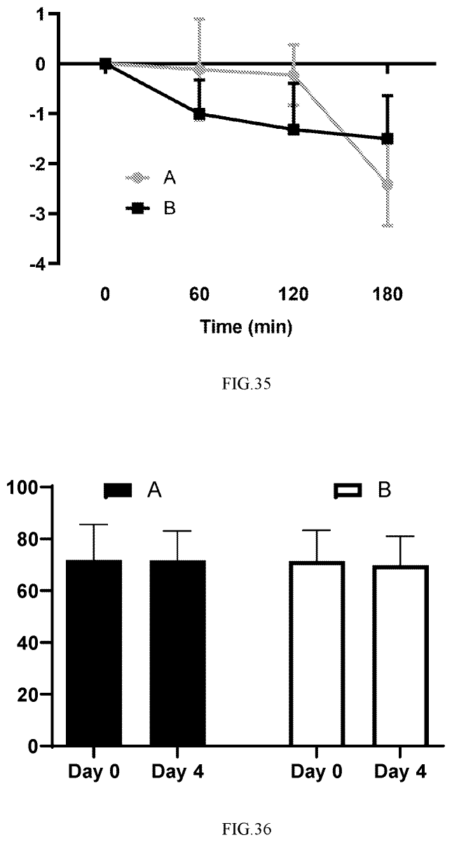

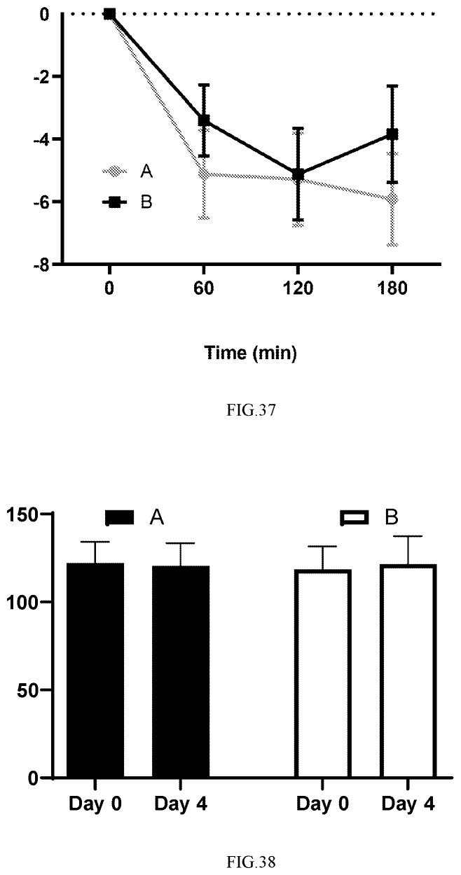

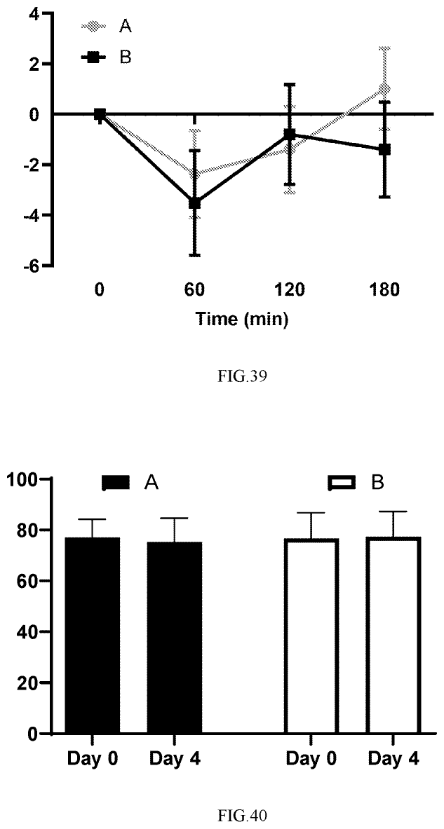

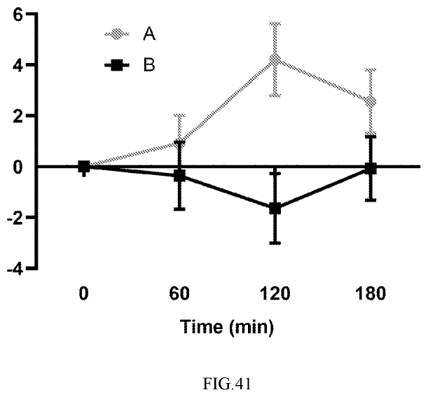

For hemodynamic parameters, systolic blood pressure, diastolic blood pressure, and heart rate were taken pre- and post-Stroop Test on Day 0, and pre- and post-Stroop Test after 4 Days of Supplementation 0, 60, 120, and 180 minutes after ingestion of 4th dose. FIGS. 38 and 39 and Tables 49-51 show exemplary data for systolic blood pressure, FIGS. 40 and 41 and Tables 52-54 shows exemplary data for diastolic blood pressure, and FIGS. 36 and 37 and Tables 55-57 show exemplary data for heart rate.

TABLE 49
Day 0 vs. Day 4 (Systolic)

| | | Group A | | Group B | | | |
|---|---|---|---|---|---|---|---|
| Variable | | Mean | (SD) | Mean | (SD) | Between-Group Effects | |
| | Day 0 | 122.0 | (12.2) | 118.6 | (12.9) | Group | 0.03 |
| | Day 4 | 120.6 | (12.8) | 121.4 | (16.0) | Time | 0.72 |

| | | | | |
|---|---|---|---|---|
| Within (p) | 0.52 | 0.27 | Group × Time | 0.13 |

*= Different from Day 0 (p < 0.05)

TABLE 50
Day 4: 0, 60, 120, and 180 minutes (systolic)

| | | A | | B | | | |
|---|---|---|---|---|---|---|---|
| Variable | Time | Mean | (SD) | Mean | (SD) | Between-Group Effects | |
| | 0 | 120.6 | (12.8) | 121.4 | (16.0) | Group | 0.914 |
| | 60 | 118.2 | (12.5) | 117.9 | (12.9) | Time | 0.101 |
| | 120 | 119.2 | (11.9) | 120.6 | (13.5) | Group × Time | 0.495 |
| | 180 | 121.6 | (11.7) | 120.0 | (12.5) | | |

| | | |
|---|---|---|
| Within Group (p) | 0.18 | 0.22 |

*= Different from 0-minute time point (p < 0.05)

TABLE 51
Changes from Baseline (systolic)

| | Mean Difference ± SE | p | 95% CI | d |
|---|---|---|---|---|
| 60 min-0 min | 1.16 ± 2.57 | 0.66 | (−4.2, 6.5) | −0.12 |
| 120 min-0 min | −0.60 ± 2.22 | 0.79 | (−5.2, 4.0) | 0.06 |
| 180 min-0 min | 2.40 ± 2.25 | 0.30 | (−2.3, 7.1) | −0.27 |

TABLE 52
Day 0 vs Day 4 (diastolic)

| | | Group A | | Group B | | | |
|---|---|---|---|---|---|---|---|
| Variable | | Mean | (SD) | Mean | (SD) | Between-Group Effects | |
| | Day 0 | 77.1 | (7.1) | 76.6 | (10.2) | Group | 0.55 |
| | Day 4 | 75.3 | (9.3) | 77.3 | (10.0) | Time | 0.56 |

| | | | | |
|---|---|---|---|---|
| Within (p) | 0.18 | 0.68 | Group × Time | 0.25 |

*= Different from Day 0 (p < 0.05)

TABLE 53
Day 4: 0, 60, 120, and 180 minutes (diastolic)

| | | PLACEBO | | DYNAMINE | | | |
|---|---|---|---|---|---|---|---|
| Variable | Time | Mean | (SD) | Mean | (SD) | Between-Group Effects | |
| | 0 | 75.3 | (9.3) | 77.3 | (10.0) | Group | 0.624 |
| | 60 | 76.2 | (9.2) | 76.9 | (8.8) | Time | 0.367 |
| | 120 | 79.5 | (11.7)* | 75.6 | (9.1) | Group × Time | 0.004 |
| | 180 | 77.8 | (9.8) | 77.2 | (9.1) | | |

| | | |
|---|---|---|
| Within Group (p) | 0.006 | 0.48 |

*= Different from 0-minute time point (p < 0.05)

TABLE 54
Changes from Baseline (diastolic)

| | Mean Difference ± SE | p | 95% CI | d |
|---|---|---|---|---|
| 60 min-0 min | 1.28 ± 1.65 | 0.45 | (−2.12, 4.68) | −0.21 |
| 120 min-0 min | 5.84 ± 1.55 | 0.001 | (2.63, 9.05) | −0.84 |
| 180 min-0 min | 2.64 ± 1.66 | 0.12 | (−0.78, 6.06) | −0.42 |

TABLE 55
Day 0 vs. Day 4 (heart rate)

| | | Group A | | Group B | | | |
|---|---|---|---|---|---|---|---|
| Variable | | Mean | (SD) | Mean | (SD) | Between-Group Effects | |
| | Day 0 | 71.8 | (13.7) | 71.4 | (11.9) | Group | 0.38 |
| | Day 4 | 71.6 | (11.4) | 69.8 | (11.2) | Time | 0.59 |

| | | | | |
|---|---|---|---|---|
| Within (p) | 0.92 | 0.45 | Group × Time | 0.58 |

*= Different from Day 0 (p < 0.05)

TABLE 56
Day 4: 0, 60, 120, and 180 minutes (heart rate)

| | | A | | B | | | |
|---|---|---|---|---|---|---|---|
| Variable | Time | Mean | (SD) | Mean | (SD) | Between-Group Effects | |
| | 0 | 71.6 | (11.4) | 69.8 | (11.2) | Group | 0.501 |
| | 60 | 66.5 | (13.5)* | 66.4 | (11.8)* | Time | <0.001 |
| | 120 | 66.4 | (13.5)* | 64.7 | (9.9)* | Group × Time | 0.421 |
| | 180 | 65.7 | (12.0)* | 66.0 | (10.9) | | |

| | | |
|---|---|---|
| Within Group (p) | <0.001 | 0.003 |

*= Different from 0-minute time point (p < 0.05)

TABLE 57
Changes from Baseline (heart rate)

| | Mean Difference ± SE | p | 95% CI | d |
|---|---|---|---|---|
| 60 min-0 min | −1.72 ± 1.46 | 0.25 | (−4.7, 1.3) | 0.27 |
| 120 min-0 min | −0.16 ± 1.58 | 0.92 | (−3.4, 3.1) | 0.02 |
| 180 min-0 min | −2.08 ± 1.69 | 0.23 | (−5.6, 1.4) | 0.53 |

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of modulating a neuroenergetic metric in a subject, comprising:
administering to the subject a composition comprising methylliberine in an amount effective to modulate the neuroenergetic metric.

2. The method of claim 1, wherein the administering is orally administering.

3. The method of claim 1, wherein the composition comprises a pharmaceutically or nutritionally acceptable carrier.

4. The method of claim 1, wherein the composition is formulated as a capsule, a powder, a buccal pouch, a nasal spray, or a tablet.

5. The method of claim 1, wherein the composition is formulated as a ready-to-drink beverage, a tea, an energy drink, or a juice.

6. The method of claim 1, wherein the methylliberine is the sole active ingredient with neuroenergetic function in the composition.

7. The method of claim 1, wherein the composition further comprises an additional active ingredient with neuroenergetic function other than caffeine and/or theacrine.

8. The method of claim 1, wherein the neuroenergetic metric is selected from the group consisting of energy, sustained energy, mental stamina, ability to focus, ability to concentrate, motivation to accomplish difficult tasks, drive, vigor, level of positive outlook, mood, feelings of well-being, and ability to tolerate stress.

9. The method of claim 1, wherein a peak of modulation of the neuroenergetic metric is at least 120 minutes post oral administration.

10. The method of claim 1, wherein a peak of modulation of the neuroenergetic metric is at least 180 minutes post oral administration.

11. A composition for modulating a neuroenergetic metric in a subject, comprising:
methylliberine in combination with a pharmaceutically or nutritionally acceptable carrier and formulated for oral administration; and
wherein the methylliberine is present in a dosage in an amount effective to modulate the neuroenergetic metric.

12. The composition of claim 11, wherein the composition is formulated as a capsule, a powder, a buccal pouch, a nasal spray, or a tablet.

13. The composition of claim 11, wherein the composition is formulated as a ready-to-drink beverage, a tea, an energy drink, or a juice.

14. The composition of claim 11, wherein the methylliberine is the sole active ingredient with neuroenergetic function in the composition.

15. The composition of claim 11, wherein the composition further comprises an additional active ingredient with neuroenergetic function other than caffeine and/or theacrine.

16. The composition of claim 11, wherein the neuroenergetic metric is selected from the group consisting of energy, sustained energy, mental stamina, ability to focus, ability to concentrate, motivation to accomplish difficult tasks, drive, vigor, level of positive outlook, mood, feelings of well-being, and ability to tolerate stress.

17. The composition of claim 11, wherein a peak of modulation of the neuroenergetic metric is at least 120 minutes post oral administration.

18. The composition of claim 11, wherein a peak of modulation of the neuroenergetic metric is at least 180 minutes post oral administration.
